(19) United States
(12) Patent Application Publication
(10) Pub. No.: US 2004/0162599 A1
(43) Pub. Date: Aug. 19, 2004

(54) Temporarily secured guidewire and catheter for use in the coronary venous system and method of using the same

(76) Inventor: Paul A. Kurth, Rancho Palos Verdes, CA (US)

Correspondence Address: Daniel L. Dawes, Myers Dawes Andras & Sherman LLP, Suite 1150, 19900 MacArthur Blvd, Irvine, CA, 92612, US

(21) Appl. No.: 10/365,890

(22) Filed: Feb. 13, 2003

(51) Int. Cl. A61N 1/05

(52) U.S. Cl. 607/122

(57) ABSTRACT

A guidewire is positioned within the coronary venous system and a pacemaker implantation catheter is arranged and configured to track the guidewire without substantially altering the position of the guidewire. A balloon catheter is deployed to temporarily and selectively secure the balloon catheter within the coronary venous system. The balloon temporarily and selectively secures or wedges the guidewire and the balloon catheter within the coronary venous system. The anchored guidewire is free along its proximal length to serve as a guide for a pacemaker lead implantation catheter. In the illustrated embodiment the movable coupling of the guidewire and catheter comprises a distally located low friction slideable coupling. The invention also includes methods for using and deploying the above apparatus in the coronary venous system.

### BACKGROUND OF THE INVENTION

1. Field of the Invention

The invention relates to the field of cardiology and in particular to the manipulation and stabilization of guidewires in the coronary venous system.

2. Description of the Prior Art

Seifert et.al., U.S. Pat. No. 5,045,061 describes a low profile, steerable balloon dilatation catheter for dilating obstructions in blood vessels is provided which also enables catheter exchanges to be performed without losing guidewire position. The system includes a guidewire and catheter that can be locked together to be operated in a manner as that of an integral wire catheter, or alternatively, they can be unlocked to allow the guidewire to be operated independently in a manner similar to that of an over-the wire catheter system. In the latter mode of operation, the length of the guidewire, at its proximal end, can be extended and the catheter can be withdrawn from the patient without causing a loss of guidewire position. With the guidewire position so maintained, a succeeding catheter can be advanced over the guidewire to the vascular location being treated.

Coelho, U.S. Pat. No. 6,251,084 discloses a system and method for effecting rapid exchange of catheters over a guidewire. A gripping mechanism is provided on one of a guide catheter and a guidewire to grip the other of the guide catheter and guidewire proximate to the distal end of the guide catheter. When engaged, the gripping mechanism inhibits movement between the guidewire and the guide catheter when a first catheter is exchanged with a second catheter over the guidewire.

Osypka, U.S. Pat. No. 5,003,990, discloses an apparatus for implanting surgical electrodes or catheters into human hearts or other hollow body organs having one or more flexible guide wires defining one or more tracks for a streamlined carriage. The rear end of the carriage is formed with a cavity for reception of the leading end of a catheter or electrode. The carriage can be pushed along the guide wire or wires by the electrode or catheter, and the leading end of thus implanted object is thereupon extracted from or expelled laterally from or beyond the cavity in the carriage while the carriage is held against forward and/or rearward movement with reference to the guide wire or wires. The carriage can be extracted from the body by the guide wire or wires or by means of a cord or hose. The carriage serves as a shield in front of the leading end of an electrode or catheter during introduction of the latter by way of an artery, a vein or another body cavity.

Mclvor et.al., U.S. Pat. No. 6,379,346, discloses an introducer is structured with an elongated tube having first and second lumens. A molded handle is coupled to the proximal end of the tube with an opening aligned with the first lumen. Further, a wall adjacent the opening of the handle defines a slot and is located diametrically opposite the second lumen. A tubular reinforcement member is located within the second lumen and the handle is provided with a second opening, thereby having a communication with the second lumen. In another embodiment a lead/catheter introducer includes an implantable lead or catheter having an elongated first lumen through which the lead or catheter passes and a second lumen extends parallel to the first lumen along at least a distal portion thereof. A tubular reinforcement member is disposed within the second lumen. The tubular reinforcement member further includes an internal lumen and a curvable stylet disposed in the lumen.

Horrigan et.al., U.S. Pat. No. 5,388,590, discloses an exchange system, a guidewire anchoring means and a method of use for catheters exchangeable over a guidewire. The guidewire anchoring means restricts movement between the guidewire and the anchoring means. The anchoring means comprises a shaft with a longitudinal lumen therethrough, the lumen being in fluid communication with a balloon disposed on the distal end of the shaft. A tubular split housing surrounds the distal end of the shaft and adheres to the balloon, the balloon being sized to expand to a larger size than the inside diameter of the split housing. The inside diameter of the split housing is sized to slidably receive a catheter, while the outside diameter of the split housing is sized to slidably fit within a guide catheter.

Christian et.al., U.S. Pat. No. 4,958,642, discloses a guide wire assembly comprising a flexible guide wire having a diameter of 0.018 inches or less and having a connector assembly. The connector assembly includes male and female connectors. The male connector includes a conductive coil spring and a core wire, which extends beyond the conductive coil. The female connector includes a recess for receiving the core wire as well as a conductive element for engaging the coil spring.

Douk et.al., U.S. Pat. No. 5,827,241, discloses a method and apparatus for withdrawing an indwelling over-the-wire catheter from an indwelling guidewire in which the guidewire is of a conventional length and is maintained in its indwelling position as the catheter is withdrawn. The position of the guidewire is maintained by constraining a segment of the catheter shaft and its included guidewire in a non-linear configuration such that the guidewire remains in its position as the catheter is withdrawn. The invention may be used to perform catheter exchanges without requiring the use of exchange wires, extension wires, rapid exchange catheters or special devices to connect the guidewire to the guide catheter.

Mazzola et.al., U.S. Pat. No. 6,443,912, discloses an apparatus for exchanging over-the-wire balloon catheters. The apparatus engages a guide wire within a guide catheter. In one embodiment the engagement is by an inflatable balloon in the guide catheter. Alternatively, the engagement is accomplished by a captivation wire in the guide catheter that has a collapsible loop portion through which the guide wire extends. Inflation of the balloon or closing the loop portion of the captivation wire within the guide catheter anchors the guide wire and restricts its movement relative to the guide catheter. Once the guide wire position is fixed, withdrawal of a first balloon catheter and subsequent introduction of a second balloon catheter over the guide wire is possible without moving the guide wire longitudinally. Thus, the positioning of the guide wire over a stenosis to be dilated is not disturbed.

Sundquist et.al., U.S. Patent Application Publication US2002/0147487, discloses a system and method for deploying medical electrical leads, which system includes a guiding device such as a guidewire used to navigate the vascular system of a body. The guiding device includes a fixation member that can be deployed to maintain the guiding device at a desired location within the vascular system. The fixation member may be an inflatable device such as a balloon, or alternatively, may be an expandable device constructed of flexible fibers that has both an expanded and a contracted state. The system may further include a coupling member located adjacent to the guiding device. The coupling member may be a rail extending distally from a proximal end of the guiding device to a point proximal the fixation member. In an alternative embodiment of the invention, the coupling member is a channel included in the body of the guiding device adapted to slidably engage an electrode assembly. The coupling member is adapted to allow the electrode assembly to be slid to the distal end of the coupling member and deployed at a predetermined implant site. In one embodiment of the invention, the coupling member is movable with respect to the guiding device. This allows the coupling member to be re-positioned to multiple implant sites to deploy more than one electrode while the fixation mechanism remains stationary within a patient's vascular system. According to yet another aspect of the invention, the guiding device includes a lumen to delivery fluoro visible medium.

### BRIEF SUMMARY OF THE INVENTION

The invention is defined as an apparatus for implanting a pacemaker lead within the coronary venous system comprising: a guidewire for positioning within the coronary venous system; an anchoring catheter arranged and configured to track the guidewire when the guidewire is disposed within the coronary venous system without substantially altering the position of the guidewire; and an anchor deployed with the anchoring catheter to temporarily and selectively secures the guidewire within the coronary venous system.

The anchor wedges the guidewire into a selected position within the coronary venous system. The anchor wedges the distal end of the anchoring catheter, the guidewire or both into a selected position within the coronary venous system.

In the preferred embodiment the anchor comprises a distal inflatable and deflatable balloon. The balloon comprises a distally positioned lateral balloon or in another embodiment, a distally located toroidal balloon.

In even broader terms the anchor comprises an expandable set of jaws, which can be actuated by an inflatable balloon or any other motive means.

The anchoring catheter is distally, movably coupled to the guidewire at a distal coupling on the catheter, and is otherwise free from the guidewire. Its distal coupling to the guidewire comprises a low friction slideable coupling such as a Teflon coated lumen or other lubricated engaging element defined in the distal end portion of the anchoring catheter.

It is also to be expressly understood that the scope of the invention also includes methods for using and deploying the above apparatus in the coronary venous system.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a diagrammatic side view of a first embodiment of the catheter and guidewire being disposed in a vein in the coronary venous system.

FIG. 2 is a diagrammatic side view of a second embodiment of the catheter and guidewire being disposed in a vein in the coronary venous system.

FIG. 3 is a diagrammatic side view of a first embodiment of the catheter and guidewire being wedged into position in a vein in the coronary venous system by a lateral balloon.

FIG. 4 is a diagrammatic side view of a second embodiment of the catheter and guidewire being wedged into position in a vein in the coronary venous system by an end balloon.

FIG. 5 is a diagrammatic side view of a third embodiment of the catheter and guidewire being wedged into position in a vein in the coronary venous system by expansion of a conical set of jaws forced open by an end balloon.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

In pacemaker lead implantations in the coronary venous system, guidewires of typically 0.018 to 0.014 inch are employed. When such guidewires are implanted into a branch vein in the coronary venous system, it is often the case that the guidewire pulls out as the pacemaker implantation catheter, which is later used to implant a pacemaker lead, is being inserted telescopically over the guidewire into the coronary venous system. The invention is thus illustrated by a separate catheter which is trackable on the guidewire and which includes a mechanism to anchor the distal end of the guidewire into position in the coronary venous system.

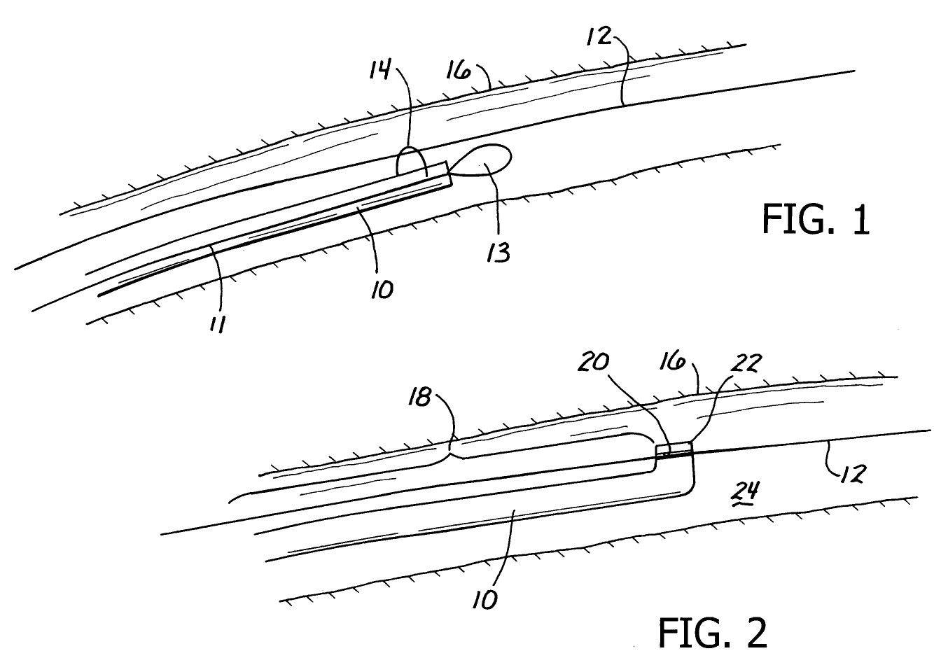

Consider first the trackability of the implantation catheter with respect to the guidewire. By trackability of the catheter it is meant that the catheter tracks to the guidewire with a minimum of force or disturbance applied to the guidewire by movement and engagement with the catheter. In other words the catheter tends to conform to the guidewire rather than the reverse. In a first embodiment, as shown in FIG. 1 catheter 10 is disposed in coronary vessel 16 to the side of guidewire 12 so that guidewire 12 is not telescopically disposed in catheter 10, but such that catheter 10 is slidingly engaged to or coupled to guidewire 12. Catheter 10 is a balloon implantation catheter and includes at least one lumen arranged and configured to allow the telescopic insertion of a balloon delivery tube 30 having an inflatable distal balloon 28 thereon for implantation into the coronary venous system. Alternatively, catheter 10 itself may have a distal balloon 28 and a lumen 30 defined therein by which fluid is supplied to distal balloon 13. Balloon 28 can be inflated in either case when positioned next to the distal end of wire 12, thereby anchoring or securing wire 12 into its implanted position in the coronary venous system. Any means now known or later devised may be employed to engage or couple guidewire 12 to catheter 10. FIG. 1 diagrammatically shows an axially extending single distal loop 14 connected to the exterior wall of catheter 10, which can be easily substituted for a short distal segment of catheter 10 having a lumen for wire 12 if desired. Once wire 12 is secured or anchored in place, then a conventional pacemaker lead (not shown) is telescopically guided over wire 12 and implanted. The anchoring of wire 12 by balloon 28 will prevent its dislodgement during pacemaker lead implantation.

FIG. 2 is a diagrammatic view of a second embodiment where catheter 10 has a guidewire lumen 20 and wherein at least a distal portion 18 of the wall of catheter 10 is removed or absent. Guidewire 12 therefore may extend through a distal or distal-most portion 22 of catheter 10 while being captured within lumen 20 in catheter 10, and then emerge into a free space 24 adjacent to catheter 10 where it is not captured within lumen 20. The longitudinal length of portions 22 and 18 can be chosen according to the application at hand, namely portion 22 is short enough not to bind or apply substantial friction force of any kind to the distal end of guidewire 12. Thus, whatever manipulations may be occurring or forces which are being created in the proximal portions of the catheter 10 between it, guidewire 12 and adjacent parts of the vascular system, will not have any appreciable effect on the stability of the distal portions of guidewire 12 which are then disposed in the coronary venous system. Note that the pacemaker lead (not shown) may be implanted using guidewire 12.

Consider now the anchorping mechanism of the invention whereby guidewire 12 is anchorped into its distal position. What is intended is that the guidewire 12 is anchorped into its position in the coronary venus system, rather than merely at some point having the catheter anchorped to the guidewire. Once catheter 10 tracks on guidewire 12 to its intended implantation position, a anchorping mechanism is activated to anchor guidewire 12 and/or catheter 10 into that anatomical position. Any anchorping mechanism now known or later devised capable of performing the anatomical anchorping is contemplated as being within the scope of the invention.

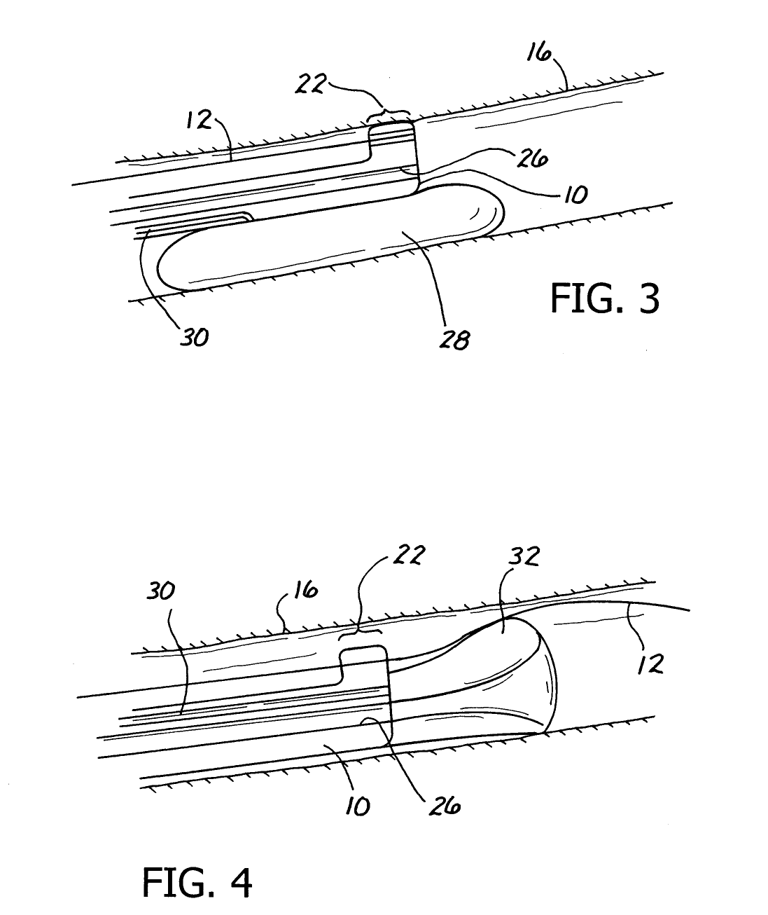

For example, in one embodiment as diagrammatically depicted in FIG. 3 catheter 10 includes an inflatable distal balloon 28 communicated to lumen 30. When at the desired anatomical position, balloon 28 is inflated by means of fluid supplied through lumen 30 wedging catheter 10 and/or guidewire 12 into vessel 16 at the desire distal implantation position. Thus, temporarily locked into position, a pacemaker lead, not shown, is on wire 12 next to catheter 10 and implanted in a conventional manner. The inflating and anchorping action of balloon 28 maintains catheter 10 and/or guidewire 12 in position as the pacemaker lead is manipulated through catheter 10 in the vascular system into the coronary venous system. In the illustrated embodiment of FIG. 3, balloon 28 is disposed laterally on the side of a distal portion of catheter 10. A circumferential end balloon 32 on catheter 10 with a central lumen in the shape of a torus is also considered as equivalent to lateral balloon 28 as depicted in FIG. 4.

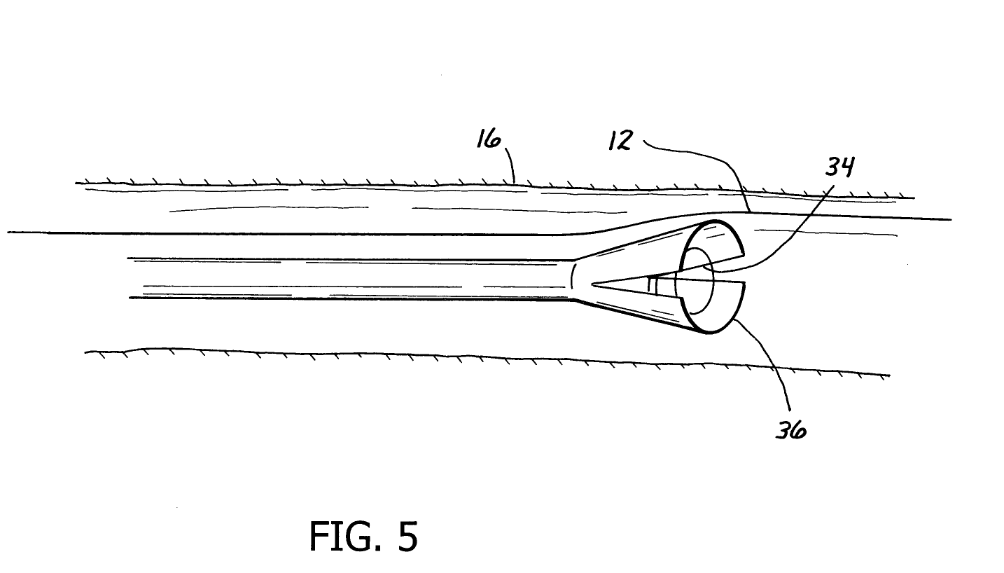

Balloon 28 or 32 is then deflated by withdrawing fluid through lumen 30. The unlocked catheter 10 may then be conveniently withdrawn while still tracking guidewire 12, followed by the withdrawal of guidewire 12 leaving the anchored pacemaker lead in place. It must be understood that any mechanical anchor or device for creating a wedging force, such as a set of expanding jaws, an expandable collar or a retractable anchor, is regarded as equivalent to the balloon. For example, instead of pressing the wire against vein 16 by means of balloon 28 or 32, the distal end of catheter 10 may be expandable to form a pair of soft opposed jaws 36 on a hinge as shown in FIG. 5. Jaws 36 are forced open when a central balloon 34 inside of jaws 36 is inflated. The exterior surface of the distal jaws 36 of catheter 10 then wedges itself in vein 16 and against wire 12 when center balloon 34 is inflated.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An apparatus for implanting a pacemaker lead within the coronary venous system comprising:
a guidewire for positioning within the coronary venous system;
an anchoring catheter arranged and configured to track the guidewire when the guidewire is disposed within the coronary venous system without substantially altering the position of the guidewire; and
an anchor deployed with the anchoring catheter to temporarily and selectively secures the guidewire within the coronary venous system.

2. The apparatus of claim 1 where the anchor wedges the guidewire into a selected position within the coronary venous system.

3. The apparatus of claim 1 where the anchor wedges the catheter and guidewire into a selected position within the coronary venous system.

4. The apparatus of claim 1 where the anchor wedges the catheter into a selected position within the coronary venous system.

5. The apparatus of claim 1 where the anchor comprises a distal inflatable and deflatable balloon.

6. The apparatus of claim 1 where the catheter has a longitudinal length and is distally, movably coupled to the guidewire at a distal coupling on the catheter, and is otherwise free from the guidewire.

7. The apparatus of claim 6 where the distal coupling of the guidewire and catheter comprises a low friction slideable coupling.

8. The apparatus of claim 5 where the balloon comprises a distally positioned lateral balloon.

9. The apparatus of claim 5 where the balloon comprises a distally located toroidal balloon.

10. The apparatus of claim 1 where the anchor comprises an expandable set of jaws.

11. A method for implanting a pacemaker lead within the coronary venous system comprising:
disposing a guidewire within the coronary venous system;
disposing an anchoring catheter by tracking the guidewire without substantially altering the position of the guidewire; and
temporarily and selectively anchoring the guidewire within the coronary venous system.

12. The method of claim 11 where temporarily and selectively anchoring the guidewire temporarily and selectively secures the guidewire by wedging the guidewire within the coronary venous system.

13. The method of claim 11 where temporarily and selectively anchoring the guidewire wedges the catheter into a selected position within the coronary venous system and traps the guidewire between the catheter and a portion of the coronary venous system.

14. The method of claim 11 where temporarily and selectively anchoring the guidewire wedges the catheter and guidewire into a selected position within the coronary venous system.

15. The method of claim 11 where temporarily and selectively anchoring the guidewire comprises inflating a distal balloon.

16. The method of claim 11 further comprising disposing a pacemaker implantation catheter by tracking the anchored guidewire.

17. The method of claim 16 where disposing an anchoring catheter by tracking the guidewire comprises coupling the guidewire and catheter with a low friction slide.

18. The method of claim 15 where inflating the distal balloon comprises inflating a distally positioned lateral balloon.

19. The method of claim 15 where inflating the distal balloon comprises inflating a distal end toroidal balloon.

20. The method of claim 11 where temporarily and selectively anchoring the guidewire comprises expanding a set of distal jaws provided on the end of the anchoring catheter.
